(19) United States
(12) Patent Application Publication
(10) Pub. No.: US 2025/0054641 A1
(43) Pub. Date: Feb. 13, 2025

(54) SYSTEM AND METHOD OF PROVIDING ARTIFICIAL INTELLIGENCE-BASED SURGICAL RESULT REPORT USING VOICE RECOGNITION PLATFORM

(71) Applicant: INDUSTRY ACADEMIC COOPERATION FOUNDATION KEIMYUNG UNIVERSITY, Daegu (KR)

(72) Inventor: Chi Heum CHO, Daegu (KR)

(21) Appl. No.: 18/687,676

(22) PCT Filed: Aug. 30, 2022
(86) PCT No.: PCT/KR2022/012924

§ 371 Date: Oct. 29, 2024

(30) Foreign Application Priority Data
Aug. 30, 2021 (KR) 10-2021-0114807

(51) Int. Cl. G16H 70/20; G16H 15/00

(52) U.S. Cl. CPC G16H 70/20; G16H 15/00

(57) ABSTRACT

A system and method of providing an artificial intelligence-based surgical result report using a voice recognition platform may receive surgical procedures and surgical diagnosis results through artificial intelligence-based voice recognition for a surgical result report written by hand after surgery, and automatically generate the surgical result report in response thereto. The present disclosure has an effect of capable of receiving surgical procedures and diagnostic results through voice recognition, configuring response selection signals with numbers to prevent inaccuracy in recognizing input values according to pronunciation, and increasing convenience and accuracy in generating a surgical result report.

### TECHNICAL FIELD

The present disclosure relates to a system and method of providing an artificial intelligence-based surgical result report, and more particularly, to a system and method of providing an artificial intelligence-based surgical result report using a voice recognition platform that receives surgical procedures and surgical diagnosis results through artificial intelligence-based voice recognition for a surgical result report written by hand after surgery and automatically generates the surgical result report in response thereto.

### BACKGROUND ART

Previously, doctors wrote surgical process and surgical result reports after surgery in hospital.

However, doctors have to write a surgical result report by hand for each surgery, so it takes a long time to write the report, and moreover, in the case of a complex surgery, the surgical result report may be written after a long time has passed since the surgery, and thus there is a disadvantage in that certain surgical processes may be omitted due to memory errors.

That is, when the surgery is complicated or the surgery time is long, doctors have to write the surgical result report after the surgery, which makes it difficult to remember and write the surgical processes thoroughly, thereby reducing the accuracy of records in the surgical result report.

### CITATION LIST

### Patent Literature

(Patent Document 1) Korean Patent No. 10-1990895

### DISCLOSURE OF INVENTION

### Technical Problem

In order to solve the foregoing problems, an aspect of the present disclosure is to provide to a system and method of providing an artificial intelligence-based surgical result report using a voice recognition platform that receives surgical procedures and surgical diagnosis results through artificial intelligence-based voice recognition for a surgical result report written by hand after surgery and automatically generates the surgical result report in response thereto.

### Technical Solution

In order to achieve the foregoing objectives, a system of providing an artificial intelligence-based surgical result report using a voice recognition platform according to a feature of the present disclosure may include:
a standard question generation unit that analyzes a standard surgical result report including surgical procedures, surgical and diagnostic results for a specific medical department to convert them into string data, and extracts keywords from the string data to respectively generate diagnostic standard question data including medical information;
a question-and-answer generation unit that extracts keywords from response areas included in the respective diagnostic standard question data;
a learning unit that learns by artificial intelligence in conjunction with an artificial neural processing network using the respective diagnostic standard question data and response data corresponding thereto as learning data; and
the respective diagnostic standard question data including response selection numbers selected in response to questions, a control unit that converts the respective diagnostic standard question data signals to transmit the converted voice signals to a user terminal, receives the response selection numbers as voice signals from the user terminal, and outputs response data corresponding to the response selection numbers through the artificial neural processing network.

A method of providing an artificial intelligence-based surgical result report using a voice recognition platform according to a feature of the present disclosure may include:
learning by artificial intelligence based on a standard surgical result report to generate a plurality of diagnostic standard question data;
the respective diagnostic standard question data including response selection numbers selected in response to questions, converting the respective diagnostic standard question data into voice signals to transmit the converted voice signals to a user terminal, and receiving the response selection numbers as voice signals from the user terminal; and
respectively generating response data corresponding to the received response selection numbers using diagnostic standard question data learned by the artificial intelligence, and generating a final surgical result report comprising surgical procedures, surgical and diagnostic results based on the generated response data.

### Advantageous Effects

With the above-described configuration, the present disclosure has an effect of capable of receiving surgical procedures and diagnostic results through voice recognition, configuring response selection signals with numbers to prevent inaccuracy in recognizing input values according to pronunciation, and increasing convenience and accuracy in generating a surgical result report.

### BRIEF DESCRIPTION OF THE DRAWINGS

FIG. 1 is a diagram showing a configuration of a system that provides an artificial intelligence-based surgical result report using a voice recognition platform according to an embodiment of the present disclosure.

FIG. 2 is a block diagram schematically showing an internal configuration of a server that provides a surgical result report according to an embodiment of the present disclosure.

FIG. 3 is a diagram showing an example of gynecological surgery procedure and diagnostic standard question data according to an embodiment of the present disclosure.

FIG. 4 is an overall conceptual diagram showing a method of providing an artificial intelligence-based surgical result report using a voice recognition platform according to an embodiment of the present disclosure.

### BEST MODE FOR CARRYING OUT THE INVENTION

Throughout the specification, when a portion may “include” a certain element, unless specified otherwise, it may not be construed to exclude another element but may be construed to further include other elements.

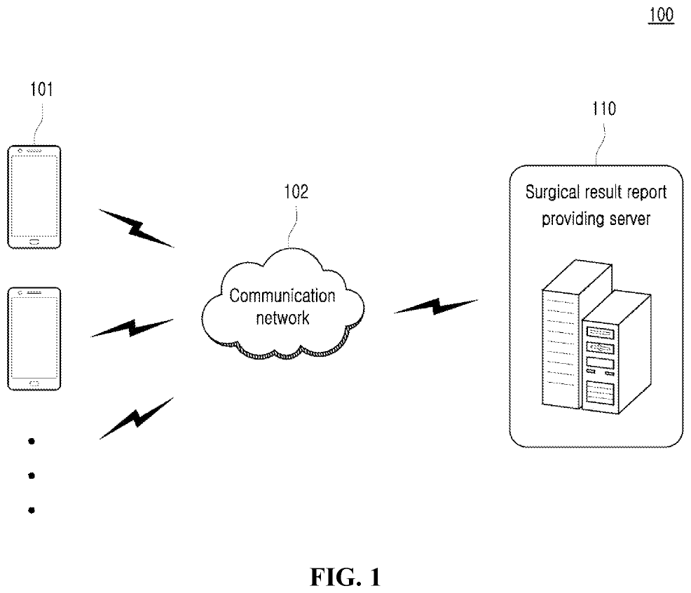

FIG. 1 is a diagram showing a configuration of a system that provides an artificial intelligence-based surgical result report using a voice recognition platform according to an embodiment of the present disclosure.

An artificial intelligence-based surgical result report providing system 100 using a voice recognition platform according to an embodiment of the present disclosure includes a user terminal 101, a communication network 102, and a surgical result report providing server 110.

The user terminal 101 includes a wired terminal or a wireless terminal equipped with a web browser based on Hypertext Markup Language (HTML) 5, and includes not only a desktop PC, which is a wired terminal, but also a mobile device such as a smartphone, a PDA, and a tablet PC.

The user terminal 101 may receive a letter, a numeral, a symbol or the like through an input module (a keyboard, a touch screen, etc.) and includes a microphone module that receives a voice signal and a speaker module that outputs a voice signal.

The user terminal 101 may provide an interface for performing communication with the surgical result report providing server 110, and for example, wireless communication such as Zigbee, RF, WiFi, 3G, 4G, LTE, LTE-A, wireless broadband Internet (WiBro), the Internet, a social network service (SNS) or the like may be used.

The user terminal 101 may store various data processed through various programs (e.g., a report generation application) and a terminal control unit (not shown), and may use a non-volatile memory such as a flash memory as a storage medium.

The user terminal 101 runs a report generation application to connect the application to the surgical result report providing server 110 through the communication network 102, and performs a series of procedures to automatically generate a surgical result report.

The communication network 102 includes both wired and wireless communication networks, and wired and wireless Internet networks may be used or linked thereto. Here, the wired network includes an Internet network such as a cable network or public switched telephone network (PSTN), and the wireless communication network includes CDMA, WCDMA, GSM, Evolved Packet Core (EPC), Long Term Evolution (LTE), and WiBro networks.

The user terminal 101 connects to the surgical result report providing server 110 to receive a report generation page, app page, program, or application for creating a surgical result report.

The surgical result report providing server 110 generates a report generation application that creates a surgical result report and transmits it to the user terminal 101 via the communication network 102.

The user terminal 101 runs a report generation application to connect to the surgical result report providing server 110.

The surgical result report providing server 110 converts the gynecological surgery procedure and diagnostic standard question data illustrated in the present disclosure into voice information, and provides the generated voice information to the user terminal 101.

The user terminal 101 receives a response selection number included in the gynecological surgery procedure and diagnostic standard question data as a user's voice signal.

The user terminal 101 transmits the received voice signal to the surgical result report providing server 110 through the communication network 102.

The surgical result report providing server 110 converts respective diagnostic standard question data into voice signals and transmits the converted voice signals to the user terminal 101.

The user terminal 101 receives the answer selection numbers included in the respective diagnostic standard question data as voice signals from the user to transmit the received voice signals to the surgical result report providing server 110.

The surgical result report providing server 110 generates response data corresponding to a voice signal received from the user terminal 101 using diagnostic standard question data learned by the artificial intelligence, and generates a final surgical result report comprising surgical procedures, surgical and diagnostic results based on the generated response data.

The diagnostic standard question data represents gynecological surgical procedures, surgical results, and diagnostic results.

An example of the diagnostic standard question data is as follows.

A first step is performed by answering a question of which platform to start with: No. 1: laparoscopy or No. 2: instrumentation. Here, the response selection number is No. 1 or No. 2.

A second step relates to which procedure is being performed, wherein a response is provided by selecting one of No. 1: hysterectomy, No. 2: myomectomy, and No. 3: ovarian cystadenoma. Here, the response selection numbers are No. 1, No. 2, and No. 3.

A third step relates to a result of diagnosis, wherein a response is provided by selecting one of No. 1: adenomyosis, No. 2: uterine myoma, No. 3: intraepithelial carcinoma, and No. 4. others. Here, the response selection numbers are No. 1, No. 2, and No. 3.

A fourth step relates to an answer to a question for checking a size of the uterus, wherein response boxes sequentially consist of No. 1 to No. 8: normal size, 6 to 8 weeks, 8 to 10 weeks, 10 to 12 weeks, 12 to 14 weeks, 14 to 16 weeks, 16 to 18 weeks, and 20 or more weeks. Here, the response selection numbers are No. 1 to No. 8.

A fifth step is performed by answering a question about whether the left and right sizes of ovaries are normal or not, respectively, wherein if they are abnormal, then one of six check boxes is selected. Here, the response selection numbers are No. 1 to No. 6.

There are simple cyst, dermoid cyst, endometrioma, mucinous, serous, and others in the six check boxes. A response is provided by selecting one of the sizes of the cyst, which are 4 cm, 6 cm, 8 cm, 10 cm, 12 cm, and 14 cm or more.

A sixth step is performed by checking for adhesions in the cervix, ovaries, fallopian tubes, and the like, wherein if there are adhesions, then a response is performed by selecting one of slight, moderate, and severe.

A seventh step relates to a question regarding the estimation of an amount of blood loss, wherein a response is provided by selecting an appropriate check box among 50 ml, 100 ml, 150 ml, 200 ml, and 300 ml or more. Here, the response selection numbers are No. 1 to No. 5.

An eighth step relates to a type of uterine surgery, wherein a response is provided by selecting one of No. 1: laparoscopic-assisted vaginal hysterectomy (LAVH) and No. 2: total laparoscopic hysterectomy (TLH). Here, the response selection number is No. 1 or No. 2.

A ninth step relates to a response to whether salpingo-oophorostomy was performed, wherein one of No. 1: unilateral salpingo-oophorectomy (USO) and No. 2: bilateral salpingo-oophorectomy (BSO) is selected. When No. 2: unilateral salpingo-oophorectomy is selected, a response must be provided whether it is right-sided or left-sided. Here, the response selection number is No. 1 or No. 2.

Each diagnostic standard question data includes a response selection number selected in response to the question.

The present disclosure may illustrate obstetrics and gynecology as an optional medical department to allow surgical procedures and diagnostic results to be input through voice recognition in a gynecology-based surgical result report, and provide a response selection signal with a number to prevent inaccuracy in recognition of input values based on pronunciation.

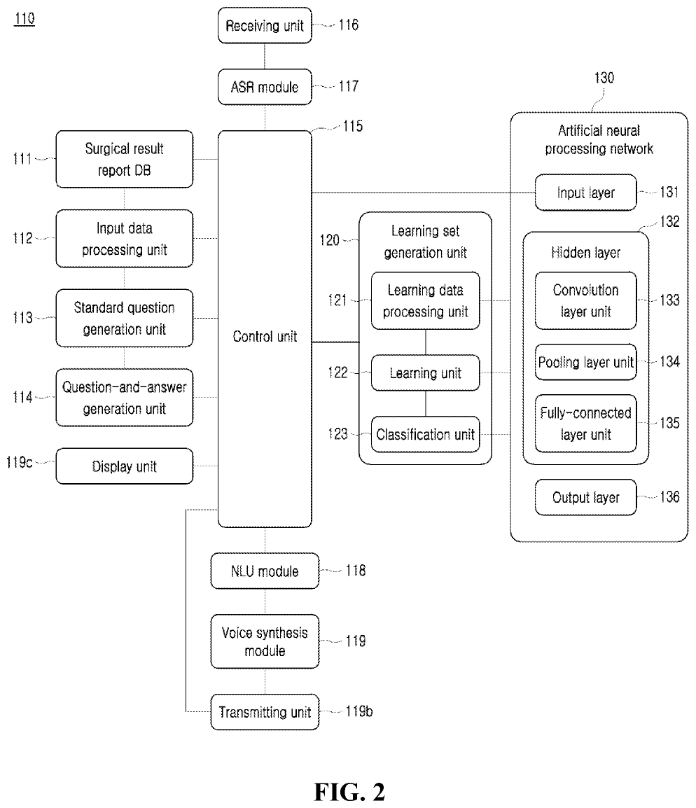

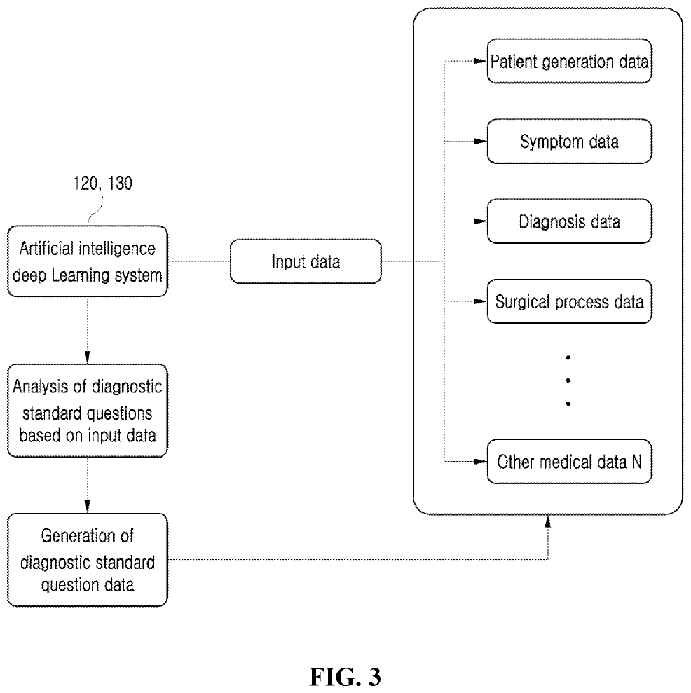

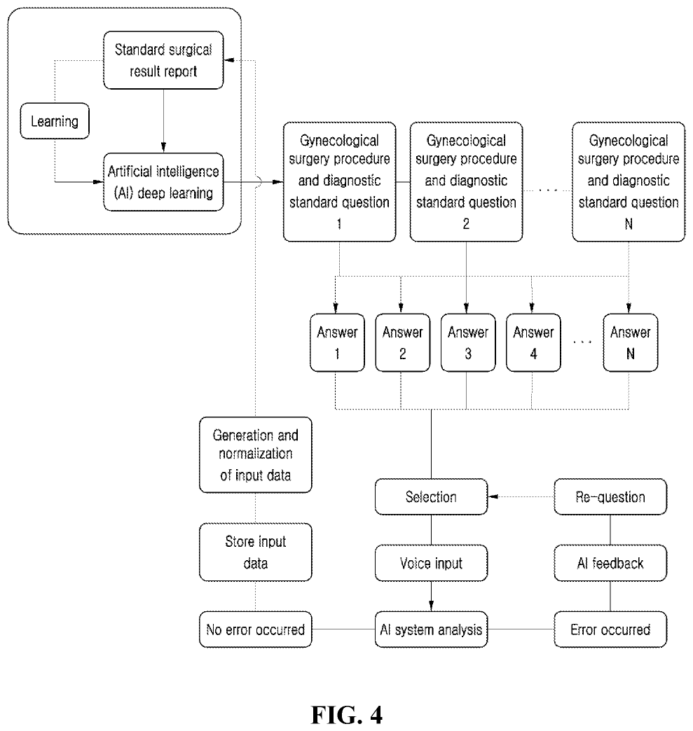

FIG. 2 is a block diagram schematically showing an internal configuration of a server that provides a surgical result report according to an embodiment of the present disclosure, FIG. 3 is a diagram showing an example of gynecological surgery procedure and diagnostic standard question data according to an embodiment of the present disclosure, and FIG. 4 is an overall conceptual diagram showing a method of providing an artificial intelligence-based surgical result report using a voice recognition platform according to an embodiment of the present disclosure.

The surgical result report providing server 110 according to an embodiment of the present disclosure includes a surgical result report database unit 111, an input data processing unit 112, a standard question generation unit 113, a question-and-answer generation unit 114, a control unit 115, a receiving unit 116, an ASR module 117, an NLU module 118, a voice synthesis module 119, a transmitting unit 119b, a display unit, a learning set generation unit 120, and an artificial neural processing network 130.

The surgical result report database unit 111 stores a plurality of standard surgical result reports according to a medical department, and performs, during storing, preprocessing on the surgical result reports to convert and store the preprocessed surgical result reports into a string format.

Upon receiving a surgical result report from the surgical result report database unit 111, the input data processing unit 112 may analyze the text sentence structure of the received surgical result report morphologically, syntactically, and semantically to calculate a vector value.

The input data processing unit 112 divides the string format, which is the text sentence structure of the surgical result report, into entities and intents by a natural language understanding (NLU) module (not shown), and processes the divided entities and intents into vector values by a vectorization module (not shown).

The NLP module may include functions such as morphological analysis, stem extraction, and stop word extraction, which are the minimum semantic units.

The vectorization module processes the divided entities and intents into a vector value using Sen2vec, Word2vec, and the like.

The standard question generation unit 113 extracts keywords from string data consisting of entities and intents in the surgical result report received from the input data processing unit 112 to generate sentence data including medical information.

The standard question generation unit 113 labels each sentence data with an index for database search to generate gynecological surgery procedure and diagnostic standard question data including medical information.

For another embodiment, the standard question generation unit 113 selects a keyword according to its frequency of appearance, and selects, when the frequency of the keyword is more than a preset number of times, the keyword as necessary while generating diagnostic standard question data.

The gynecological surgery procedure and diagnostic standard question data, which are a variety of medical data, may include patient generation data, symptom data, diagnosis data, surgical process data, and surgical result data.

The question-and-answer generation unit 114 extracts a response selection number, which is a query area, from the diagnostic standard question data, which is a question, and generates response data for the query area.

The question-and-answer generation unit 114 extracts and generates response data corresponding to the diagnostic standard question data, which is a question.

The control unit 115 receives diagnostic standard question data and response data corresponding thereto from the question-and-answer generation unit 114 to transmit the received data to the learning set generation module.

Each diagnostic standard question data includes a response selection number selected in response to the question.

The control unit 115 converts each diagnostic standard question data into a voice signal to transmit the converted voice signal to the user terminal 101, receives a response selection number as a voice signal from the user terminal 101, and outputs response data corresponding to the response selection number through the artificial neural processing network 130.

The control unit 115 generates a final surgical result report including surgical procedures, surgical and diagnostic results in consideration of the output response data.

An artificial intelligence device according to an embodiment of the present disclosure includes a learning set generation module and an artificial neural processing network 130.

The learning set generation module includes a learning data processing unit 121, a learning unit 122, and a classification unit 123.

The artificial neural processing network 130 includes an input layer 131, a hidden layer 132 consisting of a convolution layer unit 133, a pooling layer unit 134 and a fully-connected layer unit, and an output layer 136.

The learning data processing unit 121 receives a plurality of diagnostic standard question data (including indexes) and response data received from the input data processing unit 112, and distributes and stores them as learning data. The learning data processing unit 121 may be configured with a database unit capable of distributed parallel processing.

The artificial neural processing network 130 receives the diagnostic standard question data of the learning data stored in the learning data processing unit 121 into the neural network to correct errors, and outputs response data corresponding to each diagnostic standard question data using the corrected errors.

At this time, the artificial neural processing network 130 may use deep convolutional neural networks (CNNs), and may include the input layer 131, the hidden layer 132, and the output layer 136.

The input layer 131 acquires the learning data stored in the learning data processing unit 121, and stores the acquired learning data as a layer having a feature map. Here, the feature map may have a structure in which multiple nodes are arranged in two dimensions, thereby facilitating connection to the hidden layer 132, which will be described later.

The hidden layer 132 acquires a feature map of a layer located in an upper layer, and gradually extracts higher-level features from the acquired feature map. The hidden layer 132 may be configured with one or more layers and includes a convolution layer unit 133, a pooling layer unit 134, and a fully-connected layer unit.

The convolution layer unit 133, which is a component that performs a convolution operation from learning data, includes a feature map connected to a plurality of input feature maps.

A pooling layer unit 134 is configured to receive an output of the convolution layer unit 133 as an input and perform a convolution operation, that is, a sub-sampling operation, and include the same number of feature maps as that of input feature maps provided by the convolution layer unit 133 located in a lower layer of the hidden layer 132, wherein each feature map is connected one-to-one to the input feature map.

The fully-connected layer unit 135 is configured to receive an output of the convolution layer unit 133 as an input and perform learning according to an output for each category that is output from the output layer 136 to synthesize the learned local information, that is, features, so as to learn abstract content.

At this time, when the hidden layer 132 has a pooling layer unit 134, the fully-connected layer unit 135 is connected to the pooling layer unit 134 to synthesize features from the output of the pooling layer unit 134 so as to learn abstract content.

The output layer 136 maps an output for each category desired to be classified into a probability value using a function such as soft-max. At this time, a result that is output from the output layer 136 may be transmitted to the learning unit 122 or the classification unit 123 to perform error-back-propagation or may be output as response data.

The learning unit 122 performs supervised-learning, wherein the supervised-learning applies a machine learning algorithm to learning data to infer a function and finds a solution through the inferred function.

The learning unit 122 may generate a linear model representing learning data through supervised-learning, and predict a future event through the linear model.

The learning unit 122 determines how new data is classified with previously learned data based on the previously learned data.

The learning unit 122 performs learning of the artificial neural processing network 130 on gynecological surgery procedures and diagnostic standard question data, and learns response data corresponding to each diagnostic standard question data using a deep learning feature value for each type.

In one embodiment of the present disclosure, the learning of the artificial neural processing network 130 is performed through supervised-learning.

Supervised-learning is a method of receiving learning data and corresponding output data together into the artificial neural processing network 130, and updating the weights of connected edges to allow output data corresponding to the learning data to be output. For an example, the artificial neural processing network 130 of the present disclosure may update connection weights between artificial neurons using delta rules and error-back-propagation learning.

The error-back-propagation learning estimates an error through feed-forward for given learning data, and then propagates the estimated error in a reverse direction starting from the output layer 136 toward the hidden layer 132 and the input layer 131, and updates connection weights between artificial neurons to reduce the error.

The learning unit 122 calculates an error from a result acquired through the input layer 131-the hidden layer 132-the fully-connected layer unit 135-the output layer 136, and propagates the error again in the order of the output layer 136-the fully-connected layer unit 135-the hidden layer 132-the input layer 131 to correct the calculated error so as to update connection weights.

The learning unit 122 performs learning through supervised-learning to receive feature values of respective diagnostic standard question data (including the indices) that are input by using the artificial neural processing network 130, as an input vector, and generate, when passing through the input layer 131, the hidden layer 132, and the output layer 136, response data included in the respective diagnostic standard question data, as an output vector.

The learning unit 122 learns from artificial intelligence in conjunction with the artificial neural processing network 130 using the respective diagnostic standard question data and response data corresponding thereto as learning data.

The classification unit 123 outputs response data included in the diagnostic standard question data using the received response selection number.

The artificial neural processing network 130 knows in advance what input value (diagnostic standard question data) should be received and what output value (response data) should be generated.

The classification unit 123 may output the output data of the artificial neural processing network 130 having connection weights updated through error-back-propagation in the learning unit 122 as response data.

When learning data, test data, or new data not used for learning are input to the artificial neural processing network 130 having the updated connection weights, the classification unit 123 may acquire a result that is output through the input layer 131-the hidden layer 132-the fully-connected layer unit 135-the output layer 136 to output the acquired result as response data.

The classification unit 123 generates a deep learning-based classifier model through optimization based on diagnostic standard question data, response selection numbers, and response data.

The classification unit 123 outputs the received diagnostic standard question data and the response selection numbers as a result of the response data through the deep learning-based classifier model.

The receiving unit 116 receives a response selection number included in the gynecological surgery procedure and diagnostic standard question data as a user's voice signal from the user terminal 101.

The automatic speech recognition (ASR) module 117 converts the user's voice signal received from the user terminal 101 into text data.

The ASR module 117 includes a front-end speech pre-processor. The front-end speech pre-processor extracts representative features from a speech input. For example, the front-end speech pre-processor performs a Fourier transform on the speech input to extract spectral features that characterize the speech input as a sequence of representative a multi-dimensional vector. Additionally, the ASR module 117 may include one or more speech recognition models (e.g., acoustic models and/or language models) and implement one or more speech recognition engines. Examples of the speech recognition models include one of hidden Markov models, Gaussian-mixture models, deep neural network models, n-gram language models, and other statistical models.

When the ASR module 117 generates a recognition result including a text string (e.g., words, or a sequence of words, or a sequence of tokens), the recognition result is transmitted to a natural language processing module for intent inference. In some examples, the ASR module 117 generates multiple candidate text representations of the speech input. Each candidate text representation is a sequence of words or tokens corresponding to the speech input.

The natural language understanding (NLU) module 118 performs syntactic analysis or semantic analysis on each diagnostic standard question data to identify the meaning of a text string in a natural language.

Grammatical analysis divides grammatical units (e.g., words, phrases, morphemes, etc.) and identifies what grammatical elements the divided units have. The semantic analysis may be performed using semantic matching, rule matching, formula matching, and the like. Accordingly, the NLU module may acquire a domain, an intent, or a parameter necessary for the user input to express the intent.

The NLU module 118 may determine the user's intent and parameter using a mapping rule divided into a domain, an intent, and a parameter necessary for identifying the intent.

The voice synthesis module 119 converts a text string in a natural language into a voice signal. The voice synthesis module 119 uses any appropriate speech synthesis technique to generate speech outputs from text, including concatenative synthesis, unit selection synthesis, diphone synthesis, and domain-specific synthesis, formant synthesis, articulatory synthesis, hidden Markov model (HMM)-based synthesis, and sinusoidal synthesis.

The control unit 115 performs syntactic analysis or semantic analysis on each diagnostic standard question data by the natural language understanding (NLU) module 118 to identify the meaning of a text string in a natural language, and converts the text string in the natural language into a voice signal by the voice synthesis module 119.

The control unit 115 converts respective diagnostic standard question data into voice signals and transmits the converted voice signals to the user terminal 101 through the transmitting unit 119b.

In another embodiment, the control unit 115 converts the diagnostic standard question data into a voice signal according to the order of indices included therein to transmit the converted voice signal to the user terminal 101.

The user terminal 101 outputs each diagnostic standard question data as a voice signal through a speaker (not shown).

When receiving a response selection signal for each diagnostic standard question data from the user terminal 101, the surgical result report providing server 110 determines the occurrence of an error in the response selection signal in conjunction with the artificial neural processing network 130, and sets, when no error occurs, the diagnostic standard question data and response data as input data into a standard surgery result report to store the set standard surgical result report in the surgical result report database unit 111.

When receiving a response selection signal for each diagnostic standard question data from the user terminal 101, the surgical result report providing server 110 determines the occurrence of an error in the response selection signal in conjunction with the artificial neural processing network 130, and generates, when an error occurs, a re-question request signal for re-questioning the diagnostic standard question data to transmit the generated re-question signal to the standard question generation unit 113.

Although embodiments of the present disclosure have been described above in detail, the scope of rights of the present disclosure is not limited thereto, and various modifications and improvements made by those skilled in the art using the basic concept of the present disclosure as defined in the following claims also fall within the scope of the present disclosure.

### DESCRIPTION OF SYMBOLS

100: Surgical result report providing system
101: User terminal
102: Communication network
110: Surgical result report providing server

1. A system of providing an artificial intelligence-based surgical result report using a voice recognition platform, the system comprising:
a standard question generation unit that analyzes a standard surgical result report including surgical procedures, surgical and diagnostic results for a specific medical department to convert them into string data, and extracts keywords from the string data to respectively generate diagnostic standard question data including medical information;
a question-and-answer generation unit that extracts keywords from response areas included in the respective diagnostic standard question data;
a learning unit that learns by artificial intelligence in conjunction with an artificial neural processing network using the respective diagnostic standard question data and response data corresponding thereto as learning data; and
the respective diagnostic standard question data comprising response selection numbers selected in response to questions, a control unit that converts the respective diagnostic standard question data into voice signals to transmit the converted voice signals to a user terminal, receives the response selection numbers as voice signals from the user terminal, and outputs response data corresponding to the response selection numbers through the artificial neural processing network.

2. The system of claim 1, wherein the control unit generates a final surgical result report comprising surgical procedures, surgical and diagnostic results in consideration of the output response data.

3. The system of claim 1, wherein the learning unit allows feature values of the respective diagnostic standard question data to become an input vector using the artificial neural processing network, and learns, when passing through an input layer, a hidden layer, and an output layer, through supervised-learning to generate response data included in the respective diagnostic standard question data as an output vector.

4. The system of claim 1, wherein the control unit performs syntactic analysis or semantic analysis on the respective diagnostic standard question data using a natural language understanding (NLU) module to identify the meaning of a text string in a natural language, converts the text string in the natural language into a voice signal by a voice synthesis module, and transmits the converted voice signal to the user terminal.

5. The system of claim 1, wherein the control unit further comprises a receiving unit that receives response selection numbers included in the respective diagnostic standard question data as user's voice signals from the user terminal, and an automatic speech recognition (ASR) module that converts the user's voice signals received from the receiving unit into text data.

6. The system of claim 5, further comprising:
a classification unit that outputs the response selection numbers, which are the text data, as result values of the response data through a deep learning-based classifier model using the artificial neural processing network.

7. The system of claim 1, wherein the standard question generation unit respectively generates the diagnostic standard question data by labeling them with indices.

8. A method of providing an artificial intelligence-based surgical result report using a voice recognition platform, the method comprising:
learning by artificial intelligence based on a standard surgical result report to generate a plurality of diagnostic standard question data;
the respective diagnostic standard question data comprising response selection numbers selected in response to questions, converting the respective diagnostic standard question data into voice signals to transmit the converted voice signals to a user terminal, and receiving the response selection numbers as voice signals from the user terminal; and
respectively generating response data corresponding to the received response selection numbers using diagnostic standard question data learned by the artificial intelligence, and generating a final surgical result report consisting of surgical procedures, surgical and diagnostic results based on the generated response data.

9. The method of claim 8, wherein the generating of a plurality of diagnostic standard question data further comprises:
respectively generating diagnostic standard question data comprising medical information by labeling them with indices during the generation of the diagnostic standard question data.

10. The method of claim 8, further comprising:
allowing feature values of the respective diagnostic standard question data to become an input vector using an artificial neural processing network, and learning, when passing through an input layer, a hidden layer, and an output layer, through supervised-learning to generate response data included in the respective diagnostic standard question data as an output vector.
